(19) United States
(12) Patent Application Publication
(10) Pub. No.: US 2026/0005504 A1
(43) Pub. Date: Jan. 1, 2026

(54) METHOD FOR PROVIDING A GROUNDING SOLUTION FOR LIGHTNING STRIKES

(71) Applicant: Florida Power & Light Company, Juno Beach, FL (US)

(72) Inventors: Shridhar C. Nath, Falls Church, VA (US); Teodor Svaighert, Royal Palm Beach, FL (US); Phillip Raymond Thompson, Juno Beach, FL (US); Sarah Hope Daly, Juno Beach, FL (US); Sasha Lucia Sanchez, Juno Beach, FL (US); Aayan Rahim Siddiqui, Juno Beach, FL (US); Phillip Cristobal Ordonez, Juno Beach, FL (US)

(21) Appl. No.: 18/757,114

(22) Filed: Jun. 27, 2024

(51) Int. Cl. H02G 13/00; G01N 27/04; G01N 33/24; G06F 30/13

(52) U.S. Cl. CPC H02G 13/40; G06F 30/13; H02G 13/80; G01N 27/041; G01N 33/24

(57) ABSTRACT

One example includes a method for generating a grounding solution for lightning strikes. The method includes determining a geographic location of a lightning-sensitive electrical device and receiving soil resistivity data of soil at the geographic location and a surrounding geographic region. The method also includes implementing a grounding solution algorithm. The algorithm includes converting the soil resistivity data to resistance values, calculating a quantity of grounding rods for the grounding solution based on the resistance values relative to a predefined ideal resistance value, and calculating a safety distance of mounting the grounding rods with respect to the lightning-sensitive electrical device based on the resistance values. The method further includes generating installation instructions for implementing the grounding solution by mounting the grounding rods in the geographic region based on the calculated quantity of grounding rods and the calculated safety distance.

### TECHNICAL FIELD

This disclosure relates generally to power systems, and more specifically to a method for providing a grounding solution for lightning strikes.

### BACKGROUND

Renewable and natural energy sources are becoming more popular for generating power. Such renewable and natural energy sources are persistently available, require no fuel, generate no pollutants, and are more widely accepted in a more ecologically conscientious society. Such renewable and natural energy sources can be scaled to a great extent to provide renewable power plants. One such renewable power plant is a solar power system (i.e., solar field or solar farm) that harnesses a large amount of solar energy to generate electricity for a public power grid to provide clean and renewable energy to a community. A solar power system can be implemented as a large-scale photovoltaic system that includes a large number of photovoltaic modules (i.e., solar panels) arranged in series to convert light directly to electricity. By utilizing a very large number of solar panels in an open environment, a solar power system can supply power at a utility level. However, large solar power systems that are constructed in open geographic regions are subject to deleterious weather effects. Thunderstorms that produce lightning strikes can seriously damage or destroy electrical devices, such as an inverter, thereby disabling the solar power system.

### SUMMARY

One example includes a method for generating a grounding solution for lightning strikes. The method includes determining a geographic location of a lightning-sensitive electrical device and receiving soil resistivity data of soil at the geographic location and a surrounding geographic region. The method also includes implementing a grounding solution algorithm. The algorithm includes converting the soil resistivity data to resistance values, calculating a quantity of grounding rods for the grounding solution based on the resistance values relative to a predefined ideal resistance value, and calculating a safety distance of mounting the grounding rods with respect to the lightning-sensitive electrical device based on the resistance values. The method further includes generating the grounding solution for lightning strikes comprising installation instructions for implementing the grounding solution by mounting the grounding rods in the geographic region based on the calculated quantity of grounding rods and the calculated safety distance.

Another example includes a computer system. The computer system includes a user interface, a memory, and a processor. The user interface is configured to facilitate inputs and to provide outputs with respect to a user. The memory is configured to store geographic data comprising a geographic location of a lightning-sensitive electrical device and a geographic region surrounding the geographic location, and soil resistivity data of soil at the geographic location and in a plurality of locations in the geographic region. The processor is configured to execute a grounding solution algorithm. The grounding solution algorithm is configured to convert the soil resistivity data to resistance values, calculate a quantity of grounding rods based on the resistance values relative to a predefined ideal resistance value, calculate a safety distance of mounting the grounding rods with respect to the lightning-sensitive electrical device based on the resistance values, and generate a grounding solution for lightning strikes. The grounding solution includes installation instructions for mounting the grounding rods in the geographic region based on the calculated quantity of grounding rods and the calculated safety distance. The installation instructions can be provided as an output via the user interface.

Another example includes a non-transitory computer readable medium comprising machine-readable instructions. The machine-readable instructions can be executed to store geographic data comprising a geographic location of a lightning-sensitive electrical device and a geographic region surrounding the geographic location in a memory and to store soil resistivity data of soil at the geographic location and in a plurality of locations in the geographic region in the memory. The instructions can also be executed to convert the soil resistivity data to resistance values, to calculate a quantity of grounding rods based on the resistance values relative to a predefined ideal resistance value, to calculate a safety distance of mounting the grounding rods with respect to the lightning-sensitive electrical device based on the resistance values, and to generate a grounding solution for lightning strikes comprising installation instructions for mounting the grounding rods in the geographic region based on the calculated quantity of grounding rods and the calculated safety distance. The instructions can further be executed to store the grounding solution for lightning strikes in the memory, and to provide the installation instructions to a user via a user interface.

### BRIEF DESCRIPTION OF THE DRAWINGS

FIG. 1 illustrates an example of a utility power system.

FIG. 2 illustrates an example diagram of a portion of a solar field.

FIG. 3 illustrates an example block diagram of a computer system configured to implement a grounding solution algorithm.

FIG. 4 illustrates an example diagram of a visual representation of a grounding solution.

FIG. 5 illustrates another example diagram of a visual representation of a grounding solution.

FIG. 6 illustrates another example diagram of a visual representation of a grounding solution.

FIG. 7 illustrates another example diagram of a visual representation of a grounding solution.

FIG. 8 illustrates an example diagram of an implementation of a grounding solution algorithm.

FIG. 9 illustrates an example of a method for generating a grounding solution for lightning strikes.

### DETAILED DESCRIPTION

This disclosure relates generally to power systems, and more specifically to a method for providing a grounding solution for lightning strikes. As described herein, the term “grounding solution” refers to a scheme of protecting a lightning-sensitive electrical device from damage resulting from lightning strikes, the scheme including the mounting of grounding rods in the physical Earth ground in various locations of a geographic region that surrounds the lightning-sensitive electrical device. As also described herein, the term “grounding system” refers to the physical instantiation or implementation of the grounding solution that includes the tangible arrangement of the grounding rods at the various locations within the geographic region that surrounds the lightning-sensitive electrical device.

The grounding solution can be generated by implementing a grounding solution algorithm via a computer system. The grounding solution can include installation instructions for providing a grounding system for a lightning-sensitive electrical device. As described herein by example, the lightning-sensitive electrical device can correspond to an inverter in a solar power system. However, the grounding solution can be applicable to any of a variety of devices for which protection from lightning strikes is sought.

The grounding solution can be implemented by receiving geographic details regarding the lightning-sensitive electrical device. The geographic details can include a geographic location of the lightning-sensitive electrical device (e.g., latitude and longitude coordinates), as well as a surrounding geographic region. The grounding solution can also be implemented by receiving soil resistivity data associated with the geographic location and the surrounding geographic region. For example, the soil resistivity data can correspond to resistivity measurements of the soil at each of the geographic location of the lightning-sensitive electrical device and of a set of locations in the surrounding geographic region. As an example, the soil resistivity data can be provided from a variety of sources, such as from one or more enterprise organizations (e.g., a geotechnical report submitted by a surveying organization, the Soil Survey Geographic Database (SSURGO), and/or installers of the lightning-sensitive electrical device and/or associated electrical components in the geographic region). The soil resistivity data can also be provided based on estimates of resistivity values based on the types of soil in the geographic region. As another example, the soil resistivity data can include resistivity values at each of multiple depths at each of the geographic location and the locations of the geographic region.

The grounding solution algorithm can thus generate the grounding solution based on the soil resistivity data, as well as other related data. For example, the grounding solution algorithm can be configured to convert the soil resistivity data to resistance values (e.g., by averaging the resistivity values measured at each of the different depths at each of the geographic location and the separate locations of the geographic region). The grounding solution algorithm can then calculate a quantity of grounding rods necessary to implement the grounding solution based on the resistance values. For example, the calculation of the quantity of grounding rods can be based on the resistance values relative to a predefined ideal resistance value (e.g., approximately 25Ω). The grounding solution algorithm can also calculate a safety distance associated with the mounting distance of the grounding rods relative to the lightning-sensitive electrical device. As an example, the calculation of the safety distance can be based on the resistance values and lightning data, such as peak current and electric field breakdown data associated with lightning strikes. Furthermore, the grounding solution algorithm can calculate a location of the grounding rods relative to the lightning-sensitive electrical device based on mounting constraints of the grounding rods, including the safety distance and a minimum spacing between the grounding rods.

The grounding solution algorithm can thus generate the grounding solution based on the calculations associated with the grounding rods (e.g., quantity, dimensions, safety distance, minimum spacing, and/or location). The grounding solution can include installation instructions that can be provided as an output via a user interface. Accordingly, technicians can implement the grounding solution for an existing or future design of a system for which lightning protection is sought (e.g., a solar power system) by installing an associated grounding system based on the installation instructions.

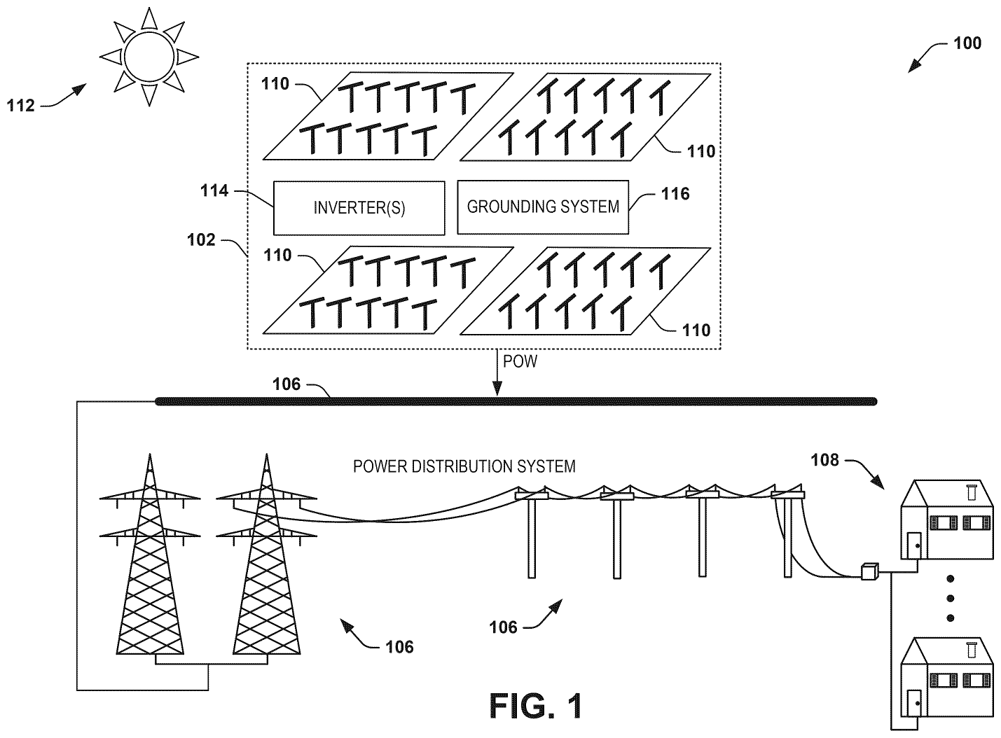

As an example, the grounding solution can be implemented in any of a variety of utility power systems, existing or a planned design, such as a solar power system as described herein, as demonstrated in the example of FIG. 1. FIG. 1 illustrates an example of a utility power system 100. The utility power system 100 includes at least one power generator system 102 that is configured to provide power, demonstrated in the example of FIG. 1 as POW, to a power transmission system 104. The power transmission system 104 can correspond to a power bus or one or more points-of-interconnect (POIs) that provide power via a power distribution system 106 (e.g., transformers, substations, and power lines) to consumers, demonstrated generally at 108. In the example of FIG. 1, the power generator system(s) 102 are demonstrated as solar power generator system(s) that include sets of solar panels 110 configured to generate the power POW via the Sun, demonstrated at 112.

In the example of FIG. 1, the power generator system(s) 102 includes one or more inverters 114 for converting the solar energy collected by the solar panels 110 to the power POW. As described herein, each of the inverter(s) 114 can correspond to a lightning-sensitive electrical device for which a grounding solution for lightning strikes is sought. To protect the inverter(s) 114 from lightning strikes that can disable or permanently damage the inverter(s), the power generator system(s) 102 can include a grounding system 116 that includes a set of grounding rods mounted in a geographic region that surrounds each of the inverter(s) 114. The grounding system 116 can be installed as a grounding solution that is generated from a grounding solution algorithm, as described herein. Accordingly, the grounding solution algorithm can be implemented on a computer system to generate the grounding solution that can be installed to provide the grounding system 116.

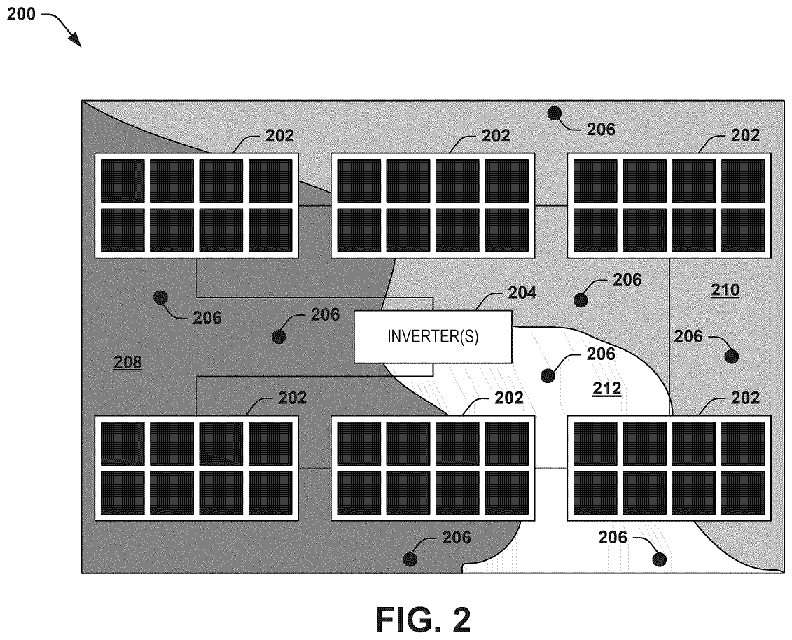

FIG. 2 illustrates an example diagram 200 of a portion of a solar power system (hereinafter “solar power system 200”). The solar power system 200 can correspond to one of the power generator system(s) 102 that includes the solar panels 110 in the example of FIG. 1. Therefore, reference is to be made to the example of FIG. 1 in the following description of the example of FIG. 2.

The solar power system 200 includes a plurality of solar panels 202 arranged in series with each other and with one or more inverters 204. As described herein, the inverter(s) 204 can correspond to a lightning-sensitive electrical device for which lightning protection is sought by implementing a grounding solution. The solar power system 200 also includes a set of grounding rods 206 that are mounted in the ground at various locations around the inverter(s) 204. Therefore, the geographic area around the inverter(s) 204 can correspond to the geographic region that surrounds the lightning-sensitive electrical device, as described herein. The grounding rods 206 can thus correspond to at least a portion of a grounding system for protecting the inverter(s) 204 from lightning strikes.

The grounding system can be provided/installed based on a grounding solution that is generated by a grounding solution algorithm, as described in greater detail herein. In the example of FIG. 2, the solar power system 200 is demonstrated as being provided on the geographic region that surrounds the inverter(s) 204 having multiple different types of soil, as indicated by the varying shades of gray. The different types of soil can include a first soil type 208, a second soil type 210, and a third soil type 212. For example, different types of soil can exhibit different resistivity values or ranges of resistivity values. The resistivity values of the soil types 208, 210, and 212 can be implemented to determine the grounding solution on which the grounding system of the solar power system 200 is based.

As an example, the resistivity values of the soil types 208, 210, and 212 can be determined in a number of ways. As an example, the resistivity values of the soil types 208, 210, and 212 can be provided from an enterprise organization, such as a service provider that measures and/or maintains information regarding the resistivity values of the soil types 208, 210, and 212 and/or specific locations of soil in the geographic region surrounding the inverter(s) 204. For example, the enterprise organization can maintain a database that defines soil types at geographic regions, including the geographic region surrounding inverter(s) 204, and/or the resistivity values of the soil types in the respective regions. An example of such an enterprise organization includes the Soil Survey Geographic Database (SSURGO). In this example, the database can merely provide soil types based on geographic coordinates, such that the resistivity values for the soil types 208, 210, and 212 can be estimated based on known resistivity values or ranges of resistivity values for different soil types as defined by the database or other information source(s).

As another example, the enterprise organization can include an organization that tests the specific resistivity values of a plurality of locations and/or a plurality of soil types in the geographic region surrounding the inverter(s) 204. An example of such an enterprise organization can include a survey company or an installation company that provides geotechnical reports (e.g., civil analysis) of the geographic region surrounding the inverter(s) 204. As yet another example, the resistivity values of the soil types 208, 210, and 212 can be obtained from direct in situ field measurements, such as part of or for the specific purpose of ascertaining the grounding solution.

Regardless of the source of the resistivity measurements of the soil types 208, 210, and 212, resistivity values of the geographic location of the inverter(s) 204 and a plurality of different locations within the geographic region surrounding the inverter(s) 204 can be implemented by a grounding solution algorithm to determine the grounding solution from which the grounding system of the solar power system 200 is based. The grounding solution, as described herein, can implement the resistivity values of the soil at the geographic location of the inverter(s) 204 as well as a plurality of other locations in the geographic region surrounding the inverter(s) 204. For example, the resistivity values can be converted to respective resistance values, which can be implemented by the grounding solution algorithm to generate the grounding solution.

In the example of FIG. 2, the grounding rods 206 are mounted at various locations in the geographic region surrounding the inverter(s) 204. The grounding rods 206 can be mounted in the locations based on the constraints determined by the grounding solution, such as included in installation instructions of the grounding solution. As an example, the quantity of grounding rods for the grounding solution, and thus the resultant grounding system, can be calculated by the grounding solution algorithm based on the resistivity values of the soil (e.g., as converted to resistance values), as well as a predefined resistance value (e.g., an ideal resistance value of one or more of the soil types 208, 210, 212).

As an example, the grounding rods 206 can be mounted based on the determination of a safety distance parameter and a minimum spacing parameter that are respectively calculated by the grounding solution algorithm. As described herein, the “safety distance parameter” or “safety distance” is defined as a minimum distance between each of the grounding rods of the grounding system (e.g., the grounding rods 206) and any lightning-sensitive electrical device (e.g., one or more inverter(s) 204 and/or the solar panels 202). As also described herein, the “minimum spacing parameter” or “minimum spacing” is defined as a minimum distance of a given one of the grounding rods of the grounding system and any other one of the grounding rods of the grounding system (e.g., the grounding rods 206). The safety distance and the minimum spacing can be the same, but are not limited to being the same.

As described in greater detail herein, the calculation of the safety distance and the minimum spacing can be based on the resistivity values of the soil (e.g., as converted to resistance values), as well as lightning data. As described herein, the term “lightning data” refers to defined electrical parameters of a lightning strike, and can further include effects of a lightning strike on the soil and/or statistical information regarding lightning strikes in a given geographic area. For example, the lightning data can include a peak current amplitude of a lightning strike, as well as an electric field breakdown value corresponding to a magnitude of an electric field that results in breakdown of the field, and thus conduction, as a function of the soil (e.g., based on a respective dielectric constant).

As a result of implementing the grounding solution algorithm described herein, a grounding solution can be generated to protect the inverter(s) 204 from lightning strikes that would damage, disable, or destroy the inverter(s) 204. The grounding solution can thus include at least each of a quantity of grounding rods, dimensions of the grounding rods, a safety distance (e.g., from a lightning-sensitive electrical device to each of the grounding rods), and a minimum distance (e.g., from a given one grounding rod to any other one grounding rod) that, when implemented as a grounding solution, can provide protection of the respective lightning-sensitive electrical device. The grounding solution can thus be implemented based on installation instructions to provide the grounding system demonstrated in the solar power system 200. Accordingly, the grounding system of the solar power system 200 can include a quantity of the grounding rods 206, a safety distance between each of the inverter(s) 204 and the grounding rods 206, and a minimum spacing between each of the grounding rods 206, as respectively calculated by the grounding solution algorithm, and thus respectively defined by the grounding solution. As a result, the grounding system can provide sufficient protection for the inverter(s) 204 from lightning strikes.

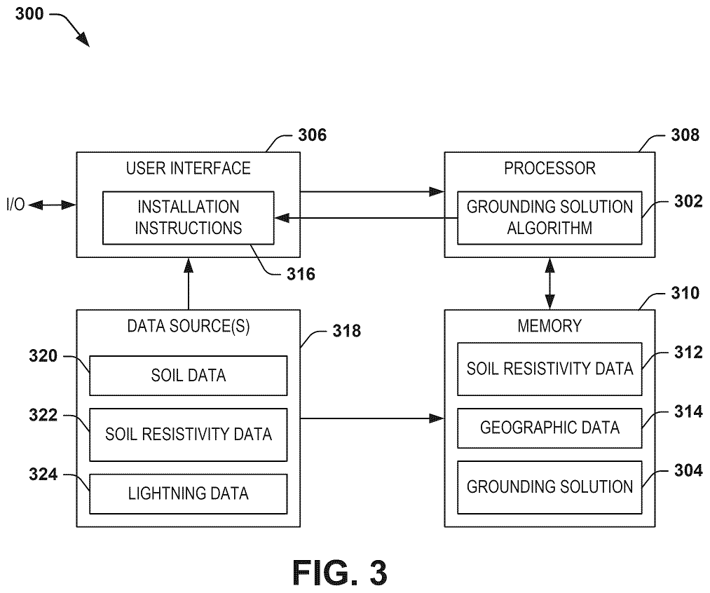

FIG. 3 illustrates an example block diagram of a computer system 300 configured to implement a grounding solution algorithm 302. As described in greater detail herein, the grounding solution algorithm 302 can be implemented to generate a grounding solution 304 that, when implemented as a grounding system, can provide lightning protection for a lightning-sensitive electrical device (e.g., the inverter(s) 204).

The computer system 300 includes a user interface 306 that can facilitate inputs and outputs (“I/O”) from and to a user. Thus, the computer system 300 can correspond to a personal computer, enterprise computer, tablet computer, dedicated terminal, or any of a variety of computer devices. The computer system 300 also includes a processor 308 configured to implement the grounding solution algorithm 302, and a memory 310 configured to store the grounding solution 304. The memory 310 is also configured to store soil resistivity data 312 and geographic data 314. The geographic data 314 can correspond to a variety of information regarding the geographic region of interest in which the grounding system is to be located. As an example, the geographic data 314 can include geographic coordinates of the geographic region of interest, and can include a specific geographic location of the lightning-sensitive electrical device. As an example, the geographic data 314 can be implemented to determine soil types associated with the geographic region, to access data from an enterprise organization data source regarding soil and/or soil resistivity measurements, to provide details regarding installation instructions 316 for the grounding solution 304, and/or to provide graphical feedback or basis for visual representations of the geographic region, such as relating the grounding solution.

In the example of FIG. 3, the computer system 300 includes at least one data source 318 that is configured to provide data to the computer system 300 as one or more inputs to the grounding solution algorithm 302. The data source(s) 318 can provide any of a variety of data that is germane to generating the grounding solution 304. As an example, the data from the data source(s) 318 can be provided directly to the memory 310, such as via accessing data from a network (e.g., a local area network (LAN), a wide area network (WAN), and/or the Internet) in response to commands provided through the user interface 306. As another example, the data from the data source(s) 318 can be provided directly to the memory 310, such as via accessing data from a network in response to automatic commands as executed by the processor 308 (e.g., via application programming interface (API) calls between the computer system 300 and the data source(s) 318). As yet another example, the data from the data source(s) 318 can be provided to the memory 310 via the user interface 306 as inputs provided by the user(s) via peripheral input devices.

In the example of FIG. 3, the data source(s) 318 can provide soil data 320, soil resistivity data 322, and/or lightning data 324. As an example, the soil data 320 and/or the soil resistivity data 322 can be provided from data source(s) 318 corresponding to enterprise organizations, such as a survey company, an installation company, and/or a geographic information database. The soil data 320 and/or the soil resistivity data 322 can be pertinent to the geographic region of interest and the geographic location of the lightning-sensitive electrical device based on the geographic data 314. The soil resistivity data 322 can be stored in the memory 310 as the soil resistivity data 312 to be accessed by the grounding solution algorithm 302.

The soil data 320 can thus correspond to soil types (e.g., the soil types 208, 210, and 212) in the geographic region of interest, as well as characteristics of the respective soil types (e.g., estimates or ranges of resistivity and/or dielectric constants of the respective soil types). The soil resistivity data 322 can be coupled with or separate from the soil data 320, and can include specific resistivity measurements of the soil of the geographic region of interest at the geographic location of the lightning-sensitive electrical device and at other locations in the geographic region surrounding the lightning-sensitive electrical device. As an example, the soil resistivity data 322 can include resistivity measurements of the soil at a given one geographic location at each of multiple depths. For example, the soil resistivity data 322 can include resistivity measurements of the soil at two or more separate depths (e.g., 3 feet, 5 feet, and 10 feet) at the geographic location of the lightning-sensitive electrical device and at each of the other locations in the geographic region for which resistivity measurements are obtained. As described herein, the soil resistivity data 322 can include measurements of electrical conductivity of soil based on the inverse relationship between resistivity and conductivity.

The lightning data 324 can correspond to a variety of a priori data associated with lightning strikes, such as a peak current amplitude. For example, the peak current amplitude can be provided as an estimate from an enterprise organization that records current amplitudes of lightning strikes and provides statistical information regarding lightning strikes. As an example, the peak current amplitude can be provided as a current amplitude value with a percent qualifier representing a likelihood of a lightning strike achieving at most the current amplitude value (e.g., 10 kA at 95%). The peak current amplitude can thus be selected, chosen, or obtained based on likelihood of achieving certain peak current amplitudes and/or to provide design considerations of the grounding solution.

As another example, the lightning data 324 can be coupled with the soil data 320, such that the lightning data 324 can also indicate electric field breakdown data. The electric field breakdown data can correspond to a maximum voltage of a lightning strike that can provide a breakdown of the electric field (and thus conduction) across soil media of the respective soil types identified by the soil data 320. For example, the electric field breakdown data can be obtained experimentally at the geographic region based on the soil or mixture of soil present along lateral directions extending linearly away from the lightning-sensitive electrical device. As another example, the electric field breakdown data can be estimated based on the soil data 320, such as based on a value or range of values of dielectric constants for different soil types defined by the soil data 320.

As yet another example, the lightning data 324 can include simulation data of the effects of historic recorded lightning strikes. The simulation data can thus provide or be extrapolated to provide the peak current amplitude and/or the lightning strike voltage as relating to electric field breakdown parameters. For example, the simulation data of the effects of historic recorded lightning strikes can be based on soil types that are comparable to the soil types defined by the soil data 320, or in geographic regions similar to the geographic region defined by the geographic data 314. Therefore, the parameters of lightning strikes that are measured at comparable sites (e.g., based on similarity in soil types/compositions) can be used to model parameters for the lightning data 324, as implemented by the grounding solution algorithm 302 to determine the grounding solution 304. Furthermore, the lightning data 324 can include historical data for a quantity or rate of lightning strikes over a given period of time, such as to enable the grounding solution algorithm 302 to provide a determination of robustness of the grounding solution 304 based on the expected frequency of lightning strikes in the geographic region (e.g., as a competing factor with respect to cost considerations).

The grounding solution algorithm 302 can thus operate to generate the grounding solution 304 based on the soil data 320, the soil resistivity data 322, and the lightning data 324. The grounding solution algorithm 302 can first convert the resistivity values of the soil resistivity data 322 into resistance values. The resistance values can be calculated via any of a variety of techniques for calculating resistance from resistivity for soil samples (e.g., the Four Point Wenner Arrangement defined by IEEE 81-2012 7.2.3). As described above, the resistivity measurements defined by the soil resistivity data 322 can include resistivity measurements at multiple depths for each geographic location. Therefore, the resistance values can correspond to a resistance that is an average of the resistivity measurements of the different depths at each geographic location. As another example, the grounding solution algorithm 302 can filter relevant resistivity data based on the spacing of electrodes for the resistivity measurements (e.g., based on a standardized multi-point lateral distance and depth). For example, the resistance R_{S} can be calculated for a specific depth, as follows:

R_{S}=ρ_{S}/(2π*D) Equation 1
Where: ρ_{S} is the resistivity measurement of the soil at the respective geographic location; and
   D is the depth for which the soil resistance R_{S} is calculated.

Accordingly, the grounding solution algorithm 302 can ascertain the resistance values that can be implemented for determination of more specific aspects of the grounding solution 304, as described herein.

As a first example, the grounding solution algorithm 302 can determine a quantity of grounding rods GR_{Q} for the grounding solution 304 for each lightning-sensitive electrical device based on the determined resistance values. For example, the grounding solution algorithm 302 can calculate the quantity of grounding rods that are necessary to achieve a desired ideal grounding resistance R_{I} (e.g., less than or approximately equal to 25Ω) based on the resistance value R_{S} of the soil. The ideal grounding resistance R_{I} can thus be selected at a low value to provide greater dispersion of the current of a lightning strike. As an example, the quantity of grounding rods GR_{Q} can be determined as follows:

GR_{Q}=(1.1*R_{S})/R_{I} Equation 2

As a second example, the grounding solution algorithm 302 can determine a safety distance D_{S} of the grounding rods relative to the lightning-sensitive electrical device. For example, the grounding solution algorithm 302 can calculate the safety distance D_{S} based on the resistance value R_{S} of the soil and based on the lightning data 324. As an example, the safety distance D_{S} (in meters) can be determined as follows:

D_{S}=(I_{PL}*R_{S})/E_{BD} Equation 3
Where:I_{PL} is a peak current amplitude of a lightning strike; and
   E_{BD} is the electric field breakdown value based on the voltage of the lightning strike.

As a third example, the grounding solution algorithm 302 can determine a minimum spacing S_{M} of the grounding rods relative to each other, such that each of the grounding rods is at least the minimum spacing away from any other grounding rod. For example, the grounding solution algorithm 302 can calculate the minimum spacing S_{M} based on the resistance value R_{S} of the soil and based on the lightning data 324. As an example, the minimum spacing S_{M} (in meters) can be determined in the same manner as the safety distance D_{S}. Thus, the safety distance D_{S} and the minimum spacing S_{M} can thus each correspond to a minimum distance that conductive components can be located in the geographic region relative to each other to mitigate electrical arcing caused by a lightning strike. Alternatively, the minimum spacing S_{M} can be calculated in another manner, as relating to the resistance value R_{S} of the soil and the lightning data 324 to ensure proper dispersion.

As a fourth example, the grounding solution algorithm 302 can determine the dimensions of the grounding rods that are implemented for the grounding solution. As an example, the dimensions of the grounding rods can be determined by the grounding solution algorithm 302 based on the soil resistivity data 322 (e.g., the soil resistance R_{S}) at a given depth of the soil and the lightning data 324. For example, the length of the grounding rods can be selected based on the depth D for which the soil resistance R_{S} is calculated, in that the grounding rods can have a length that is at least as long as the depth D. As another example, the grounding rods can have a diameter that is sufficient for dissipation of the current of a lightning strike (e.g., peak current amplitude I_{PL} of a lightning strike). Therefore, the grounding solution algorithm 302 can output quantity of grounding rods and the dimensions of the respective grounding rods as part of the grounding solution (e.g., such as included in a bill of materials (BOM) for the grounding solution) based on the input of depth-based soil resistivity data 322 and the resulting soil resistance R_{S} calculated for a given depth, and on the input of the peak current amplitude I_{PL} of a lightning strike.

The grounding solution 304 thus includes specific details regarding the implementation of mounting grounding rods in the geographic region surrounding the lightning-sensitive electrical device. As described herein, the grounding solution 304 includes the parameters of the quantity of grounding rods GR_{Q}, the safety distance D_{S} of the grounding rods relative to the lightning-sensitive electrical device, and the minimum spacing S_{M} of the grounding rods relative to each other. However, the grounding solution 304 can include any of a variety of additional components, variations, and techniques, and is thus not limited to the quantity of grounding rods GR_{Q}, the safety distance D_{S} of the grounding rods relative to the lightning-sensitive electrical device, and the minimum spacing S_{M} of the grounding rods relative to each other. An example can include a grounding solution 304 that accounts for mounting depth of the grounding rods. Furthermore, the grounding solution 304 is not limited to the arrangement of grounding rods, but can include other physical components to mitigate damage to the lightning-sensitive electrical device resulting from lightning strikes, as well.

In addition, while the grounding solution 304 is described herein as an action plan for how to protect a lightning-sensitive device and/or to provide a grounding system for existing or future lightning-sensitive electrical devices, the grounding solution 304 can include additional information. As an example, the grounding solution can include or can correspond to a determination as to whether a lightning-sensitive electrical device should or should not be installed in a given geographic region. For example, the grounding solution algorithm 302 can incorporate the soil data 320, the soil resistivity data 322, and/or the lightning data 324 to provide a grounding solution that includes a determination of difficulty of installing a grounding system for the lightning-sensitive electrical device(s). As an example, the grounding solution 304 can indicate that the soil in the geographic region is unsuitable or prohibitively expensive for installing a grounding system for a lightning-sensitive electrical device based on the soil data 320 and/or the soil resistivity data 322, or can indicate that the geographic region receives frequent lightning strikes and/or intense storms based on the lightning data 324. As a result, the grounding solution 304 can also include an indication of feasibility or difficulty (e.g., including variable cost) of installing an associated grounding system for one or more lightning-sensitive electrical devices.

The installation instructions 316 are provided to physically implement the grounding solution 304. In the example of FIG. 3, the installation instructions 316 are provided to the user interface 306 to allow a user (e.g., stakeholder) to implement the installation instructions 316. The installation instructions 316 can thus allow the grounding solution 304 to be transformed into a tangible concrete system to provide lightning protection for the respective lightning-sensitive electrical device.

As an example, the installation instructions 316 can include details as to specific hardware components are to be included in the resultant grounding system (e.g., including the quantity and dimensions of grounding rods GR_{Q}), as well as instructions for how and where to mount the grounding rods in the geographic region surrounding the lightning-sensitive electrical device. As an example, the installation instructions 316 can include the mounting constraints defined in the grounding solution 304 (e.g., the safety distance D_{S} and the minimum spacing S_{M}). Therefore, installation technicians can mount the grounding rods in any suitable location in the geographic region subject to the constraints defined by the grounding solution 304. As another example, the grounding solution algorithm 302 can be configured to determine mounting locations for the grounding rods subject to the mounting constraints. For example, the grounding solution algorithm 302 can implement a best fit algorithm that can identify one or more potential mounting arrangements for the grounding rods that each satisfy the mounting constraints defined by the grounding solution 304, as well as constraints corresponding to physical obstacles (e.g., rocks, portions of the electrical system to be protected (e.g., solar panels), and/or other prohibitive obstacles) that can be defined by the geographic data 314. Accordingly, the installation instructions 316 can provide a variety of details for how to physically implement the grounding solution 304 to instantiate the corresponding grounding system.

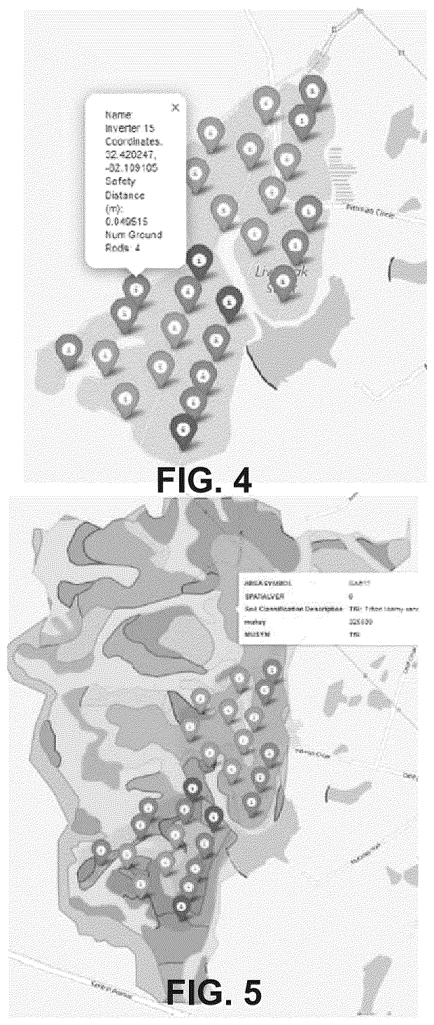

In addition, the installation instructions 316 and the user interface 306 can provide a visual representation of the grounding solution 304, such as in the context of the electrical system to be protected. The visual representation can be implemented as graphical layers that demonstrate different features of the electrical system and the grounding solution 304 on an interactive map, as demonstrated in the examples of FIGS. 4-7.

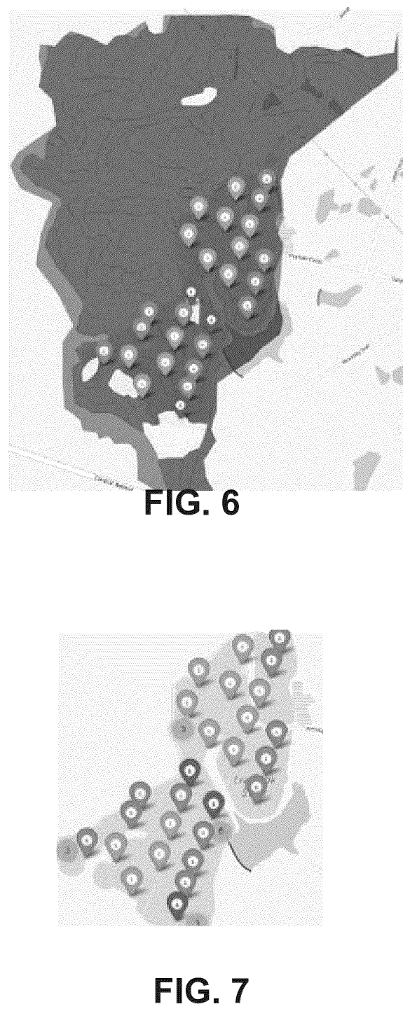

FIG. 4 illustrates an example diagram 400 of a visual representation of a grounding solution. FIG. 5 illustrates another example diagram 500 of the visual representation of the grounding solution. FIG. 6 illustrates another example diagram 600 of the visual representation of the grounding solution. FIG. 7 illustrates another example diagram 700 of the visual representation of the grounding solution. The diagrams 400, 500, 600, and 700 in the respective examples of FIGS. 4-7 can correspond to a visual representation of the grounding solution 304 that is generated via the grounding solution algorithm 302. The visual representation is demonstrated in the examples of FIGS. 4-7 as an interactive map of the geographic region corresponding to a solar power system that includes multiple lightning-sensitive electrical devices, and thus inverters by example, for which a grounding solution 304 is determined. As an example, the interactive map can be provided to one or more users via the user interface 306 (e.g., computer monitor, touchscreen, or printouts) to allow user(s) to interact with the grounding solutions 304. For example, the interactive map can be part of the installation instructions 316, or can be used for diagnostic purposes after installation of the respective grounding system.

The interactive map in the examples of FIGS. 4-7 can provide for selective activation and deactivation of layers of the visual representation of the geographic region and the grounding solution. Therefore, each of the layers can include different information that can be useful to the user(s), and can be displayed additively for selectively providing the information of each of one or more of the layers.

The diagram 400 demonstrates a first layer of the visual representation that can correspond to information regarding the inverters 402. The diagram 400 thus demonstrates the geographic location of each of the inverters 402 relative to each other and to the surrounding geographic features. Additionally, as demonstrated in the example of FIG. 4, the user(s) can select a given one of the inverters 402 to access more specific information regarding the respective inverter 402, as demonstrated by the pop-up 404. In the example of FIG. 4, the pop-up 404 demonstrates the geographic location of the respective inverter 402, as well as at least some of the details of the grounding solution for the respective inverter 402. The details of the grounding solution are demonstrated as the resistivity measurement of the soil of the geographic location of the respective inverter 402, the quantity of grounding rods, and the safety distance.

The diagram 500 demonstrates a second layer of the visual representation that can correspond to soil information regarding the geographic region surrounding the inverters 402. The soil information can correspond to a classification of the different soil types, represented in the example of FIG. 5 as different shades, in the geographic region surrounding the inverters 402. As an example, the classification of the different soil types can result from the information provided in the soil data 320, such as provided from an enterprise organization. Additionally, as demonstrated in the example of FIG. 5, the user(s) can select a portion of the geographic region to access information for the respective soil type, as demonstrated by the pop-up 502. In the example of FIG. 5, the pop-up 502 demonstrates the soil type, as well as a range of resistivity values for the respective soil type.

The diagram 600 demonstrates a third layer of the visual representation that can correspond to a choropleth map of the geographic region that represents electrical conductivity of the soil. The choropleth map can thus be representative of the inverse of the resistivity of the soil in color gradients that can be based on the range of resistivity values of the different soil types and/or the specific soil resistivity measurements at specific geographic locations in the geographic region.

The diagram 700 demonstrates a fourth layer of the visual representation that can include each of the geographic locations, demonstrated at 702, in the geographic region where specific resistivity measurements were taken (e.g., based on the soil resistivity data 322). For example, the specific resistivity measurements can be provided by an enterprise organization, such as an installation company providing a geotechnical report that includes specific in situ measurements of the resistivity of the soil in the geographic region. As an example, the user(s) can zoom in or out of the layers of the visual representation, as demonstrated by the zoom-in at 704, thereby demonstrating the geographic locations 702 where specific resistivity measurements were taken (e.g., relative to geographic locations of inverters 402). As demonstrated in the example of FIG. 7, the user(s) can interact with the fourth layer by selecting one of the geographic locations 702 in the geographic region, as demonstrated by the pop-up 706. In the example of FIG. 7, the pop-up 704 demonstrates the specific resistivity measurement at each of three separate depths at the respective geographic location 702.

The visual representation of the geographic region demonstrated in the diagrams 400, 500, 600, and 700 are provided as an example, and is not limited to the examples of FIGS. 4-7. Therefore, the visual representation can be provided to user(s) in any of a variety of ways to convey information regarding the grounding solution and/or the installed grounding system.

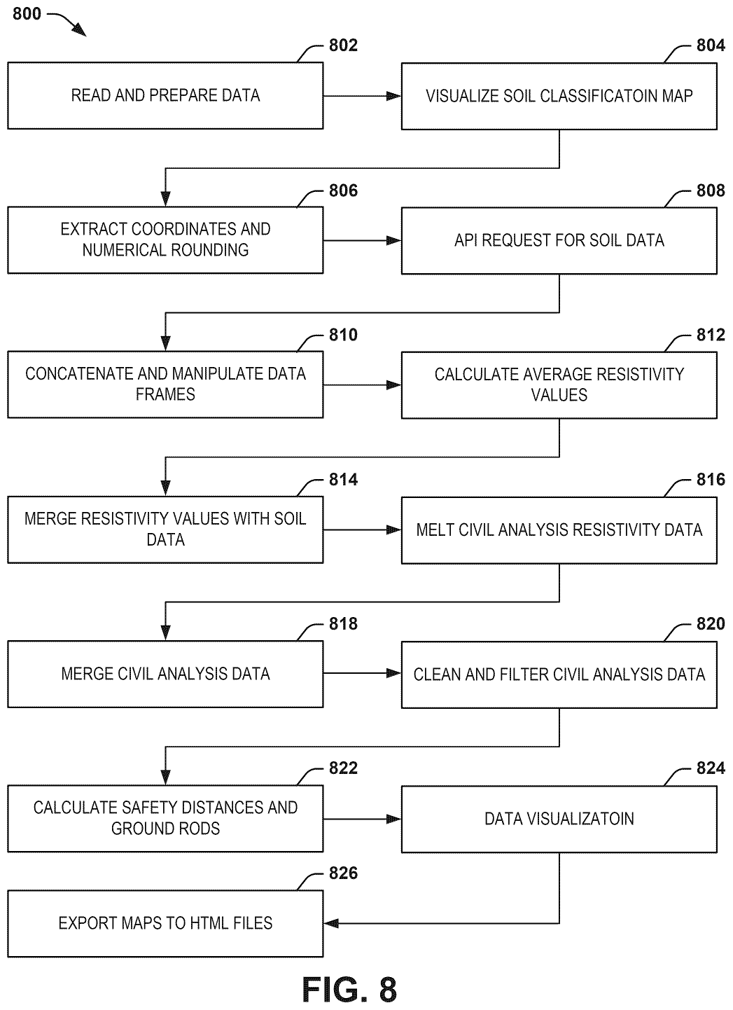

One example of generation of a grounding solution 304 via the grounding solution algorithm 302 is demonstrated in the example of FIG. 8. FIG. 8 illustrates an example diagram 800 of an implementation of a grounding solution algorithm (e.g., the grounding solution algorithm 302). The example diagram of FIG. 8 includes examples of specific computer instructions that can be implemented for executing the grounding solution algorithm, and are provided herein by example. Therefore, other examples of providing computer instructions for implementing the grounding solution algorithm can be provided instead.

At 802, the grounding solution algorithm can read and prepare data. The grounding solution algorithm can upload Environmental Systems Research Institute (ESRI) shapefiles containing soil data from an enterprise database (e.g., SSURGO) and a civil analysis spreadsheet. The grounding solution algorithm can upload ESRI shapefiles containing soil data from enterprise database and the civil analysis spreadsheet. At 804, the grounding solution algorithm can visualize the soil classification map. The grounding solution algorithm can plot the GeoDataFrame representing soil classification using a software program (e.g., matplotlib). The grounding solution algorithm can add text annotations for soil classification values on the plot. At 806, the grounding solution algorithm can extract coordinates and provide numerical rounding. The grounding solution algorithm can extract coordinates (longitude and latitude) from the polygonal geometry of the shapefile. The grounding solution algorithm can numerically round the coordinates to the nearest thousandth to reduce computational load and eliminate unnecessary precision. The grounding solution algorithm can drop duplicate coordinates from the dataset to avoid redundant API requests.

At 808, the grounding solution algorithm can provide an API request for soil data. The grounding solution algorithm can define a function to make API requests for soil data based on coordinates. The grounding solution algorithm can construct simple object access protocol (SOAP) requests with the rounded coordinates and can send them to the enterprise database API endpoint. The grounding solution algorithm can parse the XML response from the API and convert it to a DataFrame. The grounding solution algorithm can drop unwanted columns such as metadata and redundant identifiers. The grounding solution algorithm can remove duplicate rows from the DataFrame to ensure unique soil data entries. At 810, the grounding solution algorithm can concatenate and manipulate DataFrames. The grounding solution algorithm can concatenate all soil data frames obtained from the API requests into a single large DataFrame. The grounding solution algorithm can merge soil data with legend data to associate EC (Electrical Conductivity) values with soil classifications. The grounding solution algorithm can filter rows with depth within the range of 0 to 150 cm for further analysis.

At 812, the grounding solution algorithm can calculate average EC values. The grounding solution algorithm can group the filtered DataFrame by soil classification and can calculate the average EC value for each category. At 814, the grounding solution algorithm merges EC values with soil data. The grounding solution algorithm can merge the EC values DataFrame with the main soil data DataFrame based on soil classification to associate EC values with each soil entry. The grounding solution algorithm can drop unnecessary columns and can rename columns for clarity and consistency. At 816, the grounding solution algorithm can melt civil analysis resistivity data. The grounding solution algorithm can melt the civil analysis resistivity DataFrame to reshape it for easier analysis, with each row representing a unique combination of electrode spacing and key coordinates. The grounding solution algorithm can remove any extra whitespace from the “Key” column. At 818, the grounding solution algorithm can merge civil analysis data. The grounding solution algorithm can merge the key coordinates DataFrame with the melted resistivity DataFrame based on the “Key” column to associate resistivity data with each key coordinate pair.

At 820, the grounding solution algorithm can clean and filter civil analysis data. The grounding solution algorithm can clean the merged civil analysis DataFrame by removing any duplicate rows to ensure data integrity. The grounding solution algorithm can filter relevant resistivity data based on electrode spacing, retaining only the data for electrode spacings of 2.0, 5.0, and 10.0 feet. At 822, the grounding solution algorithm can calculate safety distances and grounding rods. The grounding solution algorithm can calculate the resistance for each inverter location based on resistivity data. The grounding solution algorithm can compute safety distances for lightning protection based on peak current and electric field breakdown values. The grounding solution algorithm can determine the number of grounding rods required at each location based on calculated resistance and ideal resistance values.

At 824, the grounding solution algorithm can provide data visualization. The grounding solution algorithm can create various maps using folium to visualize soil classification, EC values, resistivity data, and inverter/grounding rod locations. The grounding solution algorithm can customize maps with appropriate legends, markers, and layer controls for clarity and ease of interpretation. At 826, the grounding solution algorithm can export maps to HTML files. The grounding solution algorithm can save the generated maps as HTML files for easy sharing and viewing by stakeholders.

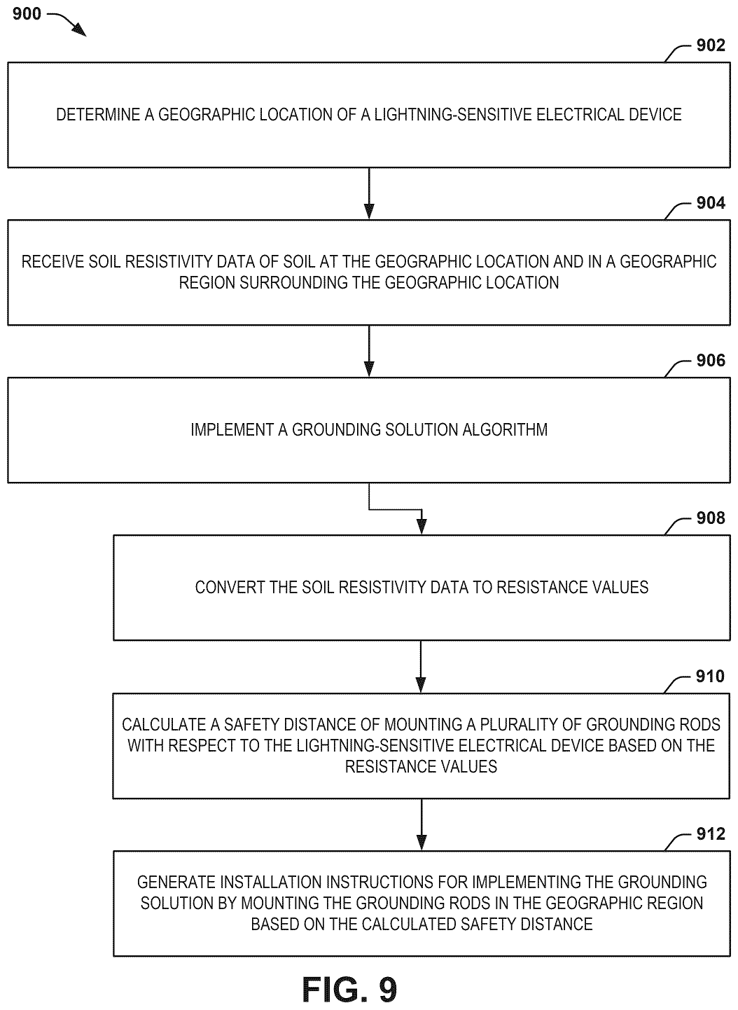

In view of the foregoing structural and functional features described above, a methodology in accordance with various aspects of the present invention will be better appreciated with reference to FIG. 9. While, for purposes of simplicity of explanation, the methodology of FIG. 9 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect of the present invention.

FIG. 9 illustrates an example of a method 900 for generating a grounding solution (e.g., the grounding solution 304) for lightning strikes. At 902, a geographic location of a lightning-sensitive electrical device (e.g., the inverter(s) 204) is determined. At 904, soil resistivity data (e.g., the soil resistivity data 322) of soil at the geographic location and in a geographic region surrounding the geographic location is received. At 906, a grounding solution algorithm (e.g., the grounding solution algorithm 302) is implemented. At 908, the grounding solution algorithm includes converting the soil resistivity data to resistance values. At 910, the grounding solution algorithm includes calculating a safety distance of mounting a plurality of grounding rods (e.g., the grounding rods 206) with respect to the lightning-sensitive electrical device based on the resistance values. At 912, the grounding solution algorithm includes generating installation instructions (e.g., the installation instructions 316) for mounting the grounding rods in the geographic region based on the calculated safety distance.

What have been described above are examples of the disclosure. It is, of course, not possible to describe every conceivable combination of components or method for purposes of describing the disclosure, but one of ordinary skill in the art will recognize that many further combinations and permutations of the disclosure are possible. Accordingly, the disclosure is intended to embrace all such alterations, modifications, and variations that fall within the scope of this application, including the appended claims. Additionally, where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, and the term “including” means including but not limited to. The term “based on” means based at least in part on.

What is claimed is:

1. A method for generating a grounding solution for lightning strikes, the method comprising:
determining a geographic location of a lightning-sensitive electrical device;
receiving soil resistivity data of soil at the geographic location and in a geographic region surrounding the geographic location;
implementing a grounding solution algorithm comprising:
converting the soil resistivity data to resistance values;
calculating a safety distance of mounting a plurality of grounding rods with respect to the lightning-sensitive electrical device based on the resistance values; and
generating installation instructions for implementing the grounding solution by mounting the grounding rods in the geographic region based on the calculated safety distance.

2. The method of claim 1, wherein implementing the grounding solution algorithm further comprises calculating a quantity of the grounding rods for the grounding solution based on the resistance values relative to a predefined ideal resistance value.

3. The method of claim 1, wherein implementing the grounding solution algorithm further comprises determining a minimum spacing between the grounding rods relative to each other based on the resistance values relative to a predefined ideal resistance value.

4. The method of claim 3, wherein implementing the grounding solution algorithm further comprises determining a location for each of the grounding rods relative to the geographic location of the lightning-sensitive electrical device based on constraints of the safety distance and the minimum spacing.

5. The method of claim 1, wherein calculating the safety distance comprises calculating the safety distance based on the resistance values and lightning data, the lightning data comprising a peak current amplitude and an electric field breakdown value.

6. The method of claim 5, wherein implementing the grounding solution algorithm further comprises determining dimensions of the grounding rods based on the resistance values and the lightning data.

7. The method of claim 1, further comprising determining soil types associated with the geographic region, wherein receiving soil resistivity data comprises estimating resistivity values of each of the determined soil types.

8. The method of claim 7, wherein converting the soil resistivity data to the resistance values comprises converting the determined resistivity values of the determined soil types to the resistance values for the grounding solution algorithm.

9. The method of claim 7, further comprising estimating lightning data associated with the geographic region based on the determined soil types and based on lightning data associated with a different geographic region having comparable soil types, the lightning data comprising a peak current amplitude and an electric field breakdown value, wherein calculating the safety distance comprises calculating the safety distance based on the resistance values and the lightning data.

10. The method of claim 1, wherein receiving soil resistivity data comprises receiving a resistivity value of soil at each of a plurality of depths at each of a plurality of other locations in the geographic region, wherein converting the soil resistivity data comprises providing a statistical aggregation of the resistivity value of the soil at each of the depths at the geographic location and at each of the depths at each of the locations in the geographic region to determine an aggregate resistivity value at the geographic location and at each of the locations in the geographic region, wherein converting the soil resistivity data comprises converting the aggregate resistivity value at each of the geographic location and at each of the locations in the geographic region to the resistance values.

11. The method of claim 1, wherein receiving soil resistivity data comprises receiving specific resistivity measurements of soil samples of a plurality of locations in the geographic region from at least one enterprise organization over a network.

12. A computer system comprising:
a user interface configured to facilitate inputs and to provide outputs with respect to a user;
a memory configured to store:
geographic data comprising a geographic location of a lightning-sensitive electrical device and a geographic region surrounding the geographic location;
soil resistivity data of soil at the geographic location and in a plurality of locations in the geographic region; and
a processor configured to execute a grounding solution algorithm, the grounding solution algorithm being configured to:
convert the soil resistivity data to resistance values;
calculate a quantity of grounding rods based on the resistance values relative to a predefined ideal resistance value;
calculate a safety distance of mounting the grounding rods with respect to the lightning-sensitive electrical device based on the resistance values; and
generate a grounding solution for lightning strikes comprising installation instructions for mounting the grounding rods in the geographic region based on the calculated quantity of grounding rods and the calculated safety distance, the installation instructions being provided as an output via the user interface.

13. The system of claim 12, wherein the grounding solution algorithm is further configured to determine a minimum spacing between the grounding rods relative to each other based on the resistance values relative to the predefined ideal resistance value.

14. The system of claim 13, wherein the grounding solution algorithm is further configured to determine a location for each of the grounding rods relative to the geographic location of the lightning-sensitive electrical device based on constraints of the safety distance and the minimum spacing.

15. The system of claim 12, wherein the grounding solution algorithm is configured to calculate the safety distance based on the resistance values and lightning data, the lightning data comprising a peak current amplitude and an electric field breakdown value.

16. The system of claim 15, wherein the memory is further configured to store a classification of soil types associated with the geographic region, wherein the grounding solution algorithm is further configured to at least one of:
estimate resistivity values of each of the soil types; and
estimate the lightning data associated with the geographic region based on the determined soil types and based on separate lightning data associated with a different geographic region having comparable soil types.

17. A non-transitory computer readable medium comprising machine-readable instructions, the machine-readable instructions being executed to:
store geographic data comprising a geographic location of a lightning-sensitive electrical device and a geographic region surrounding the geographic location in a memory;
store soil resistivity data of soil at the geographic location and in a plurality of locations in the geographic region in the memory;
convert the soil resistivity data to resistance values;
calculate a quantity of grounding rods based on the resistance values relative to a predefined ideal resistance value;
calculate a safety distance of mounting the grounding rods with respect to the lightning-sensitive electrical device based on the resistance values;
generate a grounding solution for lightning strikes comprising installation instructions for mounting the grounding rods in the geographic region based on the calculated quantity of grounding rods and the calculated safety distance;
storing the grounding solution for lightning strikes in the memory; and
providing the installation instructions to a user via a user interface.

18. The medium of claim 17, wherein the machine-readable instructions are further executed to determine a minimum spacing between the grounding rods relative to each other based on the resistance values relative to the predefined ideal resistance value.

19. The medium of claim 18, wherein the machine-readable instructions are further executed to determine a location for each of the grounding rods relative to the geographic location of the lightning-sensitive electrical device based on constraints of the safety distance and the minimum spacing.

20. The medium of claim 17, wherein the machine-readable instructions are executed to calculate the safety distance based on the resistance values and lightning data, the lightning data comprising a peak current amplitude and an electric field breakdown value.

21. The medium of claim 20, wherein the machine-readable instructions are further executed to:
store a classification of soil types associated with the geographic region in the memory;
estimate resistivity values of each of the soil types; and
estimate the lightning data associated with the geographic region based on the determined soil types and based on separate lightning data associated with a different geographic region having comparable soil types.
